(19) United States
(12) Patent Application Publication
(10) Pub. No.: US 2006/0129131 A1
(43) Pub. Date: Jun. 15, 2006

(54) Catheter

(76) Inventors: Ulrich J. Pfeiffer, Munchen (DE); Daniel Muller, Pfaffing (DE)

Correspondence Address: WILLIAM COLLARD;COLLARD & ROE, P.C., 1077 NORTHERN BOULEVARD, ROSLYN, NY, 11576, US

(21) Appl. No.: 10/540,077

(22) PCT Filed: Dec. 19, 2003
(86) PCT No.: PCT/EP03/51079

§ 371 Date: Nov. 22, 2005

(30) Foreign Application Priority Data
Dec. 23, 2002 (DE) 102607613

(51) Int. Cl. A61M 25/01

(52) U.S. Cl. 604/528

(57) ABSTRACT

Catheter (1), comprising a catheter body (2), the inner volume of which forms a first catheter channel (4), which serves to accommodate a guide wire (15) during the introduction of the catheter into the body of a patient, with at least one separating wall (5), arranged therewithin, which divides off at least one further catheter chamber (6) in the interior. According to the invention, a catheter chamber with the largest possible cross-sectional area with a given external diameter may be provided, whereby the catheter body (2) has a tubular outer wall (3), the cross-sectional area (F2) of the further catheter chamber (6) is smaller than the cross-sectional area (F1) of the first catheter chamber (4) and the further catheter chamber (6) is arranged such as to comprise a wall section which is part of the tubular outer wall (3).

The invention relates to a catheter having a catheter body, the interior of which forms a first catheter lumen, which serves to accommodate a guide wire during the introduction of the catheter into the body of a patient, having at least one partition disposed in the interior, which divides off at least one further catheter lumen in the interior.

Catheters having two or more lumens are used in surgical interventions and in intensive care medicine, in order, for example, to measure temperature and pressure in various body regions, to take samples of liquid for analysis purposes, or to supply liquids.

Catheters for invasive measurement of temperature and blood pressure have a round lumen for measuring pressure, supplying and removing liquids, as well as a D-shaped, half-moon-shaped, or round lumen for accommodating a thermistor for the invasive temperature measurement.

The known catheters of this type cannot be used for small blood vessels, because of their diameter dimension. When their outside diameter is reduced, the flow resistance increases markedly, so that the supply and removal of liquid and even the pressure measurement are significantly impaired. In particular, if the catheter is equipped with a comparatively long catheter tube, the great flow resistance becomes noticeable to a disruptive degree.

The invention is based on the task of creating a catheter of the type stated initially, which is characterized by a small outside diameter and, at the same time, a low flow resistance in at least one lumen.

This task is accomplished, in the case of a catheter of the type stated initially, in that the catheter body has a tubular outer wall and that the cross-sectional area of the further catheter lumen is smaller than the cross-sectional area of the first catheter lumen, and the further catheter lumen is disposed in such a manner that it has a wall section that is part of the tubular outer wall.

By means of this method of arrangement, according to the invention, the cross-sectional area of the catheter tube is utilized in optimal manner, so that the larger catheter lumen, in any case, has such a cross-sectional area that its flow resistance lies within limits that permit problem-free pressure measurement with a low response delay, even with extremely small outside diameters. The catheter is suitable for invasive temperature and blood pressure measurements (e.g. in connection with the determination of the heart/time volume, wherein the catheter is introduced into an artery, for example), even in children. Despite the essentially eccentric placement of the further catheter lumen, the catheter demonstrates sufficient stiffness so as to allow handling of the catheter in the usual manner. The catheter according to the invention can also be provided with a longer catheter body, in comparison with the state of the art, without causing problems in the pressure measurement, and without exceeding the diameter values of conventional catheters.

Surprisingly, it has been shown that despite the cross-sectional area of the first catheter lumen, which deviates from the circular shape, it is excellently suited for accommodating the guide wire, and that the catheter can be pushed into a blood vessel over the guide wire, without problems, for example according to the Seldinger technique, until it has reached its final position. The subsequent “drawing” of the guide wire also proceeds without problems, and in particular, no jamming or wedging of the guide wire in the first catheter lumen occurs, and the latter is subsequently used, for example, to measure blood pressure or to supply liquid substances, in accordance with its intended purpose.

In an advantageous embodiment, the cross-sectional area of the first catheter lumen and the cross-sectional area of the further catheter lumen have a common axis of symmetry in the cross-sectional plane, and the quotient of the cross-sectional area of the first catheter lumen and the cross-sectional area of the further catheter lumen is greater than the square of the quotients of the width of the first catheter lumen, measured along the common axis of symmetry, and the width of the further catheter lumen, measured along the common axis of symmetry. If this regulation for dimensions is adhered to, it is assured that the cross-sectional area of the first catheter lumen has an optimal size.

It is furthermore advantageous if the partition runs in arc shape over at least one section of the same. A partition configured in this manner molds itself to the shape of the further lumen and thereby permits the best possible utilization of the available space, whereby it is particularly advantageous if the arc-shaped partition has a convex side that faces the first catheter lumen, and a concave side that faces the further catheter lumen.

The choice of an approximately round shape for the further catheter lumen has proven itself to be particularly suitable not only from the aspect of an optimal division of space, but also from the aspect of achieving a sufficient bending stiffness of the catheter body.

The further catheter lumen is particularly suitable for accommodating a temperature sensor that can be disposed in the region of the catheter tip, and which can fill the available cross-sectional area preferably by four-fifths, in order to achieve a play that allows easy insertion, or also completely.

If polyurethane, preferably having a Shore hardness between 60D and 85D, is used as the material for the catheter, it is found that on the one hand, the catheter demonstrates satisfactory stiffness, and on the other hand, reliable anti-lock sliding of the catheter relative to the guide wire disposed in the first catheter lumen is also possible.

With regard to reliable sliding of the guide wire, it has proven to be advantageous if the guide wire has a diameter that amounts to 65% to 95% of the distance between the partition and the outer wall.

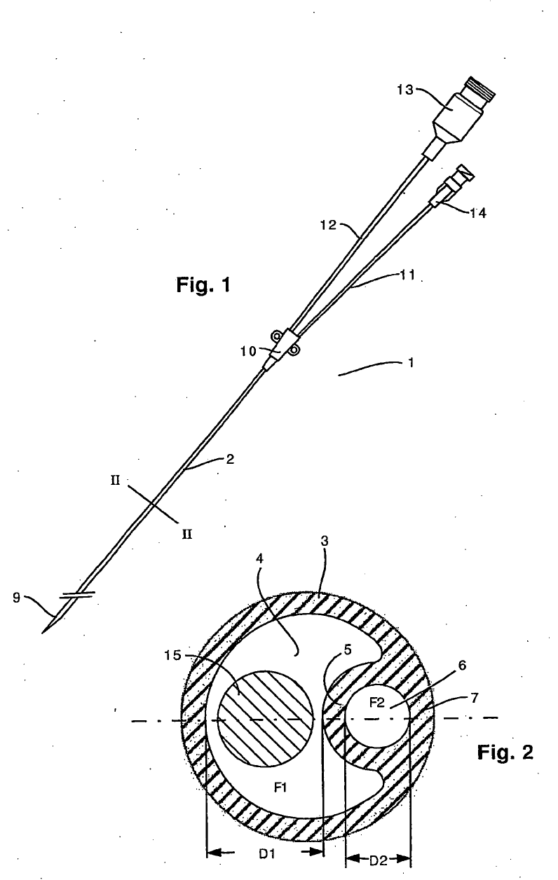

The invention will be explained in greater detail below, on the basis of an exemplary embodiment shown schematically in FIGS. 1 and 2.

FIG. 1 shows an external view of the catheter according to the invention,

FIG. 2 shows a cross-section of the catheter along the section line II-II in FIG. 1, on an enlarged scale.

The thermodilution catheter shown in FIG. 1, for continuous measurement of temperature and pressure in a blood vessel (e.g. femoral artery) has a catheter body 2 that extends from a Y connector piece 10 to the catheter tip 9. On the side of the Y connector piece 10 opposite the catheter body, the former is connected with a pressure tube 11 as well as with an electrical line 12. A plug 13 is disposed at the end of the electrical line, making a connection to an evaluation unit (not shown) possible. A tube coupling piece 14 is located at the end of the pressure tube 11.

The structure of the catheter body 2 according to the invention can be seen in the cross-sectional drawing of FIG. 2.

The catheter body 2 has a tubular outer wall 3 having an approximately uniform wall thickness. A first catheter lumen 4 is configured in the interior of the catheter body 2, which lumen is sickle-shaped in cross-section, as can be seen in the figure. The interior contains a partition 5 that divides off a further catheter lumen 6, which is approximately circular, as can be seen in the figure. The cross-sectional area F1 of the sickle-shaped first catheter lumen 4 is greater than the cross-sectional area F2 of the further catheter lumen 6.

The further catheter lumen 6 is disposed eccentrically, in such a manner that it has a wall section 7 in common with the outer wall 3. The remaining wall of the further catheter lumen 6, by means of which the two catheter lumens 4 and 6 are separated, i.e. the partition 5, is arc-shaped. The concave side of the arc-shaped partition 5 faces the (round) further catheter lumen 6, the convex side faces the (sickle-shaped) first catheter lumen 4. In the exemplary embodiment described, the further catheter lumen 6 serves to accommodate a temperature sensor. The further catheter lumen 6 can also be intended to accommodate an optical fiber sensor (not shown). Depending on the application case, the further catheter lumen 6 is sealed or open at the catheter tip 9.

By means of the structure described, solid material regions are avoided, to the greatest possible extent, and thereby the collapse regions that often occur after extrusion, because of such material accumulations, are also avoided.

In the case of the division of the interior of the catheter according to the invention, as described, a comparatively large cross-sectional area is imparted to the first lumen, so that the flow resistance can be kept low, if, for example, blood flows through this catheter lumen. This allows a reduction of the catheter diameter as compared with conventional catheters, or, alternatively, the use of significantly longer catheter bodies with the same diameter.

The dimensions of the two catheter lumens and the arrangement of the catheter lumens are selected in such a manner that the cross-sectional area F1 of the first catheter lumen 4 and the cross-sectional area F2 of the further catheter lumen 6 have a common axis of symmetry in the cross-sectional plane, and the quotient of the cross-sectional area F1 of the first catheter lumen 4 and the cross-sectional area F2 of the further catheter lumen 6 is greater than the square of the quotients of the width D1 of the first catheter lumen 4, measured along the common axis of symmetry, and the width D2 of the further catheter lumen 6, measured along the common axis of symmetry. Consequently, the following applies:

$$\frac{F 1}{F 2} > {( \frac{D 1}{D 2} )}^{2}$$

As is further evident from FIG. 2, a guide wire 15 made of steel is located in the further catheter lumen 4. The further catheter lumen 4 therefore serves as a guide wire lumen during the introduction phase of the catheter. As is evident from the figure, the diameter of the guide wire 15 is less than the clear width D1 between the outer wall 3 and the peak of the arc-shaped partition 5.

1. Catheter (1) having a catheter body (2), the interior of which forms a first catheter lumen (4), which serves to accommodate a guide wire (15) during the introduction of the catheter into the body of a patient, having at least one partition (5) disposed in the interior, which divides off at least one further catheter lumen (6) in the interior,
wherein the catheter body (2) has a tubular outer wall (3) and the cross-sectional area (F2) of the further catheter lumen (6) is smaller than the cross-sectional area (F1) of the first catheter lumen (4), and the further catheter lumen (6) is disposed in such a manner that it has a wall section (7) that is part of the tubular outer wall (3).

2. Catheter according to claim 1, wherein the cross-sectional area (F1) of the first catheter lumen (4) and the cross-sectional area (F2) of the further catheter lumen (6) have a common axis of symmetry in the cross-sectional plane, and the quotient of the cross-sectional area (F1) of the first catheter lumen (4) and the cross-sectional area (F2) of the further catheter lumen (6) is greater than the square of the quotients of the width (D1) of the first catheter lumen (4), measured along the common axis of symmetry, and the width (D2) of the further catheter lumen (6), measured along the common axis of symmetry.

3. Catheter according to claim 1, wherein the partition (5) runs in arc shape over at least one section of same.

4. Catheter according to claim 3, wherein the arc-shaped partition (5) has a convex side that faces the first catheter lumen (4), and a concave side that faces the further catheter lumen (6).

5. Catheter according to claim 1, wherein the cross-sectional area (F1) of the first catheter lumen (4) has a rounded sickle shape.

6. Catheter according to claim 1, wherein the cross-sectional area (F2) of the further catheter lumen (6) is round.

7. Catheter according to claim 1, wherein a temperature sensor is disposed in the further catheter lumen (6).

8. Catheter according to claim 7, wherein the temperature sensor is disposed in the vicinity of the catheter tip (9).

9. Catheter according to claim 6, wherein the cross-sectional area of the temperature sensor fills the cross-sectional area of the further catheter lumen (6) by at least four-fifths.

10. Catheter according to claim 7, wherein the cross-sectional area of the temperature sensor fills the cross-sectional area of the further catheter lumen (6) completely.

11. Catheter according to claim 1, wherein an optical fiber sensor is disposed in the further catheter lumen (6).

12. Catheter according to claim 1, wherein the further catheter lumen (6) is open in the region of the catheter tip.

13. Catheter according to claim 1, wherein the further catheter lumen (6) is closed in the region of the catheter tip.

14. Catheter according to claim 1, wherein the catheter body is made from plastic having a Shore hardness of 60D to 85D.

15. Catheter according to claim 14, wherein the plastic is polyurethane.

16. Catheter system according to claim 1, having a guide wire (15), wherein the guide wire (15) has a diameter that amounts to 65% to 95% of the distance (D1) between the partition (5) and the outer wall (3).
